(19) United States
(12) Patent Application Publication
(10) Pub. No.: US 2026/0345946 A1
(43) Pub. Date: Nov. 5, 2026

(54) SLEEP SYSTEM ADJUSTMENTS BASED ON USER DATA FROM SENSORS IN PHYSICAL CONTACT WITH A USER

(71) Applicant: T-Mobile USA, Inc., Bellevue, WA (US)

(72) Inventors: Kavitha Swaminathan, Olathe, KS (US); Christopher Bailey, Overland Park, KS (US)

(21) Appl. No.: 19/195,085

(22) Filed: Apr. 30, 2025

(51) Int. Cl. A61M 21/02; A47G 9/10; A61B 5/00; A61G 7/018

(52) U.S. Cl. CPC A61M 21/02; A47G 9/1045; A61B 5/6892; A61B 5/7267; A61G 7/018; A47G 9/1036; A61M 2205/3303; A61M 2205/3584; A61M 2230/30

(57) ABSTRACT

Described herein are a user device and sleep system. The user device determines health information for a user based on user data captured by a sensor physically contacting the user and provides the health information to a sleep system. The sleep system receives the health information and adjusts a setting of the sleep system based on the health information. The user data may include at least one of a heartrate, blood pressure, an electrocardiogram reading, an oxygen level, a temperature, or a respiratory rate. The sleep system may be at least one of a mattress or pillow, and the setting may be at least one of a firmness setting, an elevation angle setting, a thickness setting, or a temperature setting.

### BACKGROUND

With each new generation, user devices, such as mobile devices and Internet-of-Things (IoT) devices (e.g., smart watches) are able to measure more and more user health metrics. IoT devices now measure heartrate, blood oxygen levels, and blood pressure. Based on these and other sensed data, IoT devices can even perform analyses such as electrocardiograms.

At the same time, health measurements and actions are taken by many items that surround the user. For example, smart mattresses and smart pillows-often referred to as “sleep systems” can listen to a sleeper disposed on them, detecting snoring or breathing patterns from audio sensors and adjusting mattress levels based on this information.

Along with greater abilities to measure things about a user that may indicate the user's health and, in some instances, to take complementary actions to help alleviate medical issues, technology is able to do so in a more informed and intelligent way through the use of artificial intelligence.

### BRIEF DESCRIPTION OF THE DRAWINGS

The detailed description is set forth with reference to the accompanying figures. In the figures, the left-most digit(s) of a reference number identifies the figure in which the reference number first appears. The same reference numbers in different figures indicate similar or identical items.

FIG. 1 is an overview diagram showing a user device in physical contact with a user to determine health information for the user and a sleep system connected to the user device to receive the health information and adjust a setting of the sleep system based on the health information.

FIG. 2 is a flow diagram of an illustrative process for a user device to determine health information for a user based on user data captured by a sensor physically contacting the user and to provide the health information to a sleep system, such as a mattress or pillow, to cause the sleep system to adjust a setting.

FIG. 3 is a flow diagram of an illustrative process for a sleep system to receive health data for a user captured by a sensor physically contacting the user and to adjust a setting of the sleep system based on the health information.

FIG. 4 is a schematic diagram of a computing device capable of implementing functionality of at least one of the user device or sleep system described herein.

### DETAILED DESCRIPTION

This disclosure is directed in part to a user device and sleep system. The user device may determine health information for a user based on user data captured by a sensor physically contacting the user and may provide the health information to a sleep system. The sleep system may receive the health information and may adjust a setting of the sleep system based on the health information.

The user device may be an Internet-of-Things (IoT) device or a user equipment (UE) connected to an IoT device and the user data may include at least one of a heartrate, blood pressure, an electrocardiogram reading, an oxygen level, a temperature, or a respiratory rate. In some examples, the IoT device may be a watch, a ring, a necklace, an earring, an ear cuff, or other piece of jewelry or apparel worn on a hand, wrist, arm, neck, upper torso, or head. In other examples, the IoT device may be a medical device (e.g., a glucose monitor) paired to a UE.

The sleep system may be at least one of a mattress or pillow, and the setting may be at least one of a firmness setting, an elevation angle setting, a thickness setting, or a temperature setting. In some examples, the sleep system may also or instead include a heated blanket, and its setting may be a temperature setting. The sleep system may connect to the user device to receive health information-which may be instructions or information helping determine instructions. The sleep system may also verify that the user is on the sleep system before performing any adjustments. In some examples, the sleep system may be a guest sleep system (e.g., at a hotel) that is able to interface with multiple, different user devices to provide different adjustments for different users.

In various implementations, an artificial intelligence (AI) component included in the user device, the sleep system, or an external device may contribute to determining health information for a user or adjustments to sleep system settings. The AI component may include a large language model (LLM) that is trained on the user's data or data of other users to enable recognition of health patterns for the user. The AI component may also improve in its determination of health information or setting adjustments through continued monitoring of user data. If one adjustment to sleep system settings produces “better” user data at later measurements than another, the AI component may increasingly cause the sleep system to select the adjustment with better results. “Better,” as used herein with respect to user data, may refer to user data that fits health thresholds or ranges defined by a configuration as optimal for users in general, or for the user in particular.

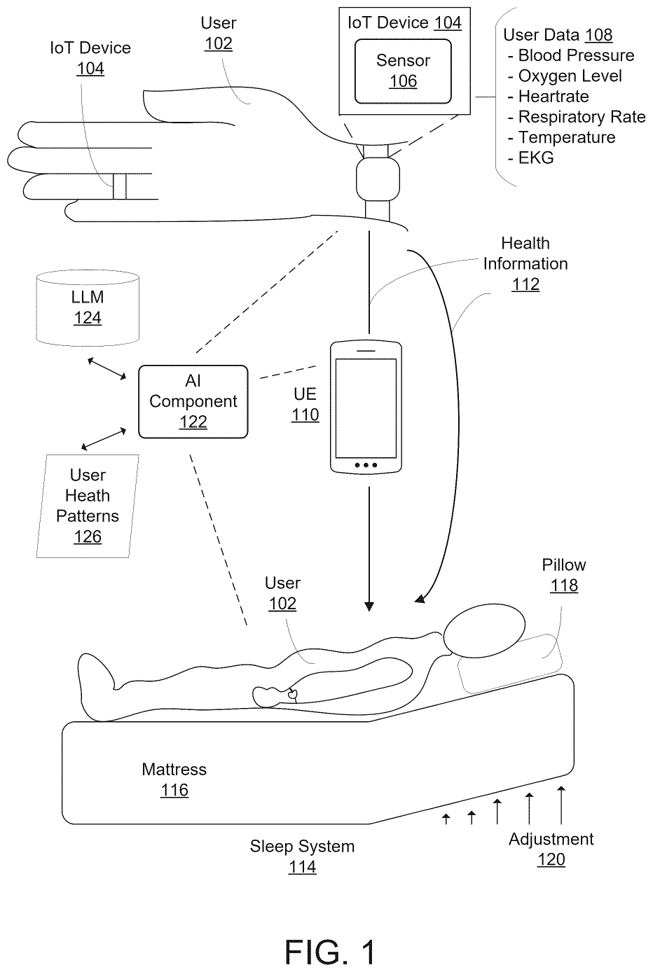

FIG. 1 is an overview diagram showing a user device in physical contact with a user to determine health information for the user and a sleep system connected to the user device to receive the health information and adjust a setting of the sleep system based on the health information. As illustrated, a user 102 may have one or more IoT devices 104 equipped with sensor(s) 106 that are in physical contact with the user 102 (e.g., around a wrist, on a finger, etc.) and capture user data 108 (e.g., heartrate, blood pressure, oxygen level, temperature, respiratory rate etc.). In some examples, the user 102 may also have a UE 110 in proximity to the user. Either directly from the IoT device(s) 104 or indirectly through the UE 110, health information 112 for the user 102 determined based on the user data 108 may be sent to a sleep system 114. The sleep system 114 may include, for example, a mattress 116, a pillow 118, or both. Based on the health information 112, the sleep system 114 may adjust a setting of the sleep system 114 (shown in FIG. 1 as “adjustment 120”). The adjustment 120 may be an adjustment to a temperature, a firmness, a thickness, or an elevation angle. Further, an AI component 122 may be part of any one or more of the IoT device(s) 104, the UE 110, or the sleep system 114. The AI component 122 may reference an LLM 124, may determine health patterns 126 for the user 102, and may participate in determining health information 112 based on the LLM 124, health patterns 126, and user data 108.

In various implementations, the IoT device(s) 104 may be any sort of wearable devices capable of communication with the UE 110, the sleep system 114, and/or a telecommunications network. Connections with the UE 110 and sleep system 114 may be WiFi connections or Bluetooth connections, and a connection with a telecommunications network may be cellular, WiFi, or Bluetooth. To enable such connections, the IoT device(s) 104 may each have one or more radio antennae for sending and receiving wireless transmission. Further, for cellular transmissions at least, the IoT device(s) 104 may use any generation of radio access technology. Also, the telecommunications networks mentioned herein may include both terrestrial networks and non-terrestrial networks (e.g., those associated with satellites).

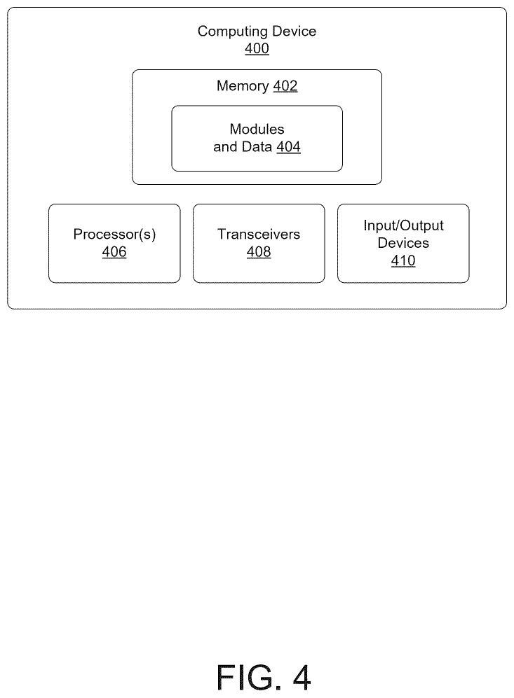

In some examples, such as those shown in FIG. 1, the IoT device(s) 104 may include a watch (also referred to as a “smart watch”) and a ring. While IoT device(s) 104 as a class may include a wide variety of devices, such as glasses, goggles, necklaces, earring, ear cuffs, hats, canes, apparel, etc., the IoT device(s) 104 described herein may be those that have sensor(s) 106 capable of capturing user data 108 such as heartrates, blood oxygen, blood pressure, temperature, and respiratory rate when worn. These IoT device(s) 104 may also have logic, such as a health platform or health applications, capable of deriving some types of user data 108 (e.g., electrocardiogram) from other user data 108 and, in some examples, of generating health information 112 from the user data 108. Such health information 112 can include the user data 108 itself, instructions based on the user data 108, health patterns, etc. The IoT device(s) 104 may also include the AI component 122 or part of the AI component 122. In other examples, however, both determining of health information 112 and AI functions may be done by another device (e.g., UE 110) and the IoT device(s) 104 may simply determine and provide the user data 108. An example IoT device 104 is illustrated in FIG. 4 and described further herein with reference to that figure.

In various implementations, the UE 110 may be any sort of wireless communications device, such as a cellular phone, a tablet computer, a laptop, etc. The UE 110 may be capable of receiving the user data 108, health information 112, or both and may determine health information 112 or participate in determining health information 112. As previous noted, the health information 112 can include the user data 108 itself, instructions based on the user data 108, health patterns, etc. In some examples, the UE 110 may also include the AI component 122 or part of the AI component 122. When receiving or determining health information 112, the UE 110 may provide that health information to a sleep system 114. An example UE 110 is illustrated in FIG. 4 and described further herein with reference to that figure.

The mattress 116 and pillow 118 may each include the materials and fabrics traditionally associated with mattresses and pillows or may, instead or also, include newer materials. In addition, the mattress 116 and pillow 118 may also include electrical and/or mechanical components that allow for adjustments 120. Components may warm or cool the mattress 116 and pillow 118, may make the mattress 116 and pillow 118 firmer or softer, may make mattress 116 and pillow 118 thinner or thicker, or may adjust an elevation angle-for instance, a bottom portion of the mattress 116, where user 102's feet may go, may bend upwards at an angle with respect to the remainder of the mattress 116 in response to a higher blood pressure on the part of the user 102.

In addition to electrical or mechanical components for making adjustments 120, the mattress 116 and pillow 118 may each include electrical components, such as memory and a processor, for receiving and processing health information 112 to determine adjustments 120 based on the health information 112. The adjustments 120 made by the mattress 116 or pillow 118 may be adjustments to a firmness setting, an elevation angle setting of part of the mattress 116 or pillow 118, an elevation angle setting of all of the mattress 116 or pillow 118, a thickness setting of the mattress 116 or pillow 118, or a temperature setting of the mattress 116 or pillow 118, which then drive mechanical components of the mattress 116 or pillow 118 to actuate the changed setting. In some examples, the mattress 116 and pillow 118 may also include the AI component 122 or part of the AI component 122. An example sleep system 114/mattress 116 and pillow 118 is illustrated in FIG. 4 and described further herein with reference to that figure.

In various implementations, in addition to user data 108, other user data, such as images or sounds, may be used to determine the health information 112. Such images or sounds may be captured by the IoT device(s) 104, by the UE 110, or by the sleep system 114. When captured by the sleep system 114, the sleep system 114 may have camera and/or microphone for capturing images or sound, which may then be processed by the sleep system 114, along with the health information 112, to determine further and/or different adjustments 120. For example, by detecting sounds, the sleep system 114 may determine that a user 102 has sleep apnea and may raise a head portion of the mattress 116 (where the user's head is laid) at an angle with respect to a remainder of the mattress 116. Snoring sounds could also or instead by captured by the IoT device(s) 104 and/or UE 110.

In some implementations, before making an adjustment 120, the sleep system 114 may verify that a user 102 is on the sleep system 114 (e.g., laying on the mattress 116 and/or pillow 118). The sleep system 114 may be equipped with sensors (e.g., a pressure sensor, a temperature sensor, a weight sensor, etc.) or may be informed by the IoT device(s) 104 of the user's presence.

Further, the sleep system 114 may accept connections from the IoT device(s) 104 or UE 110. In some examples, the sleep system 114 may be a guest sleep system of a hotel or similar establishment and may accept connections and health information from multiple different IoT devices 104 or UEs 110. For example, the sleep system 114 may be a mattress 116 with a QR code that the UE 110 can scan. Scanning can result in a connection used by an IoT device 104, the UE 110, and the guest mattress 116 to exchange health information 112 and make adjustments 120.

As noted previously, the AI component 122 may be implemented among one or more of the IoT device(s) 104, the UE 110, and the sleep system 114. The AI component 122 may be any sort of AI and may utilize an LLM 124. The LLM 124 may be trained on user data 108, user data from other users, or both, and based on the training may determine health pattern(s) 126 for the user 102. Such health pattern(s) 126 may represent expected values in the captured user data 108 based on historic values, predefined healthy value ranges, etc. Thus, if the user 102 typically has a very low heartrate, as represented in the health pattern(s) 126, and the user data 108 registers a mildly elevated heart rate from that experience among a typical population, the AI component 122 may determine health information 112 (e.g., instructions) to make an adjustment 120. For other users without the health pattern(s) 126 showing the very low heartrate, no adjustment 120 might be made.

An AI component 122 may also learn user preferences-if a user 102 reverse an adjustment 120 of the sleep system 114, the AI component 122 may learn not to determine health information 112 leading to a similar adjustment 120 in the future.

Further, the AI component 122 or other logic of the IoT device(s) 104, UE 110, or sleep system 114 may prioritize some adjustments 120 for some health conditions over others, and the configuration for determining adjustment(s) 120 to make may be received, e.g., from a remote information source.

In various implementations, the AI component 122 may continue to evaluate further user data 108 after the sleep system 114 makes an adjustment 120 to determine whether the user data 108 improves relative to health patterns 126 and health standards. When there is no or insufficient improvement, the AI component 122 may determine different health information 112 resulting in a different adjustment 120 and monitor and evaluate its effects, too. Over time, the AI component 122 may optimize the adjustments 120 chosen for different sorts and magnitudes of user data 108.

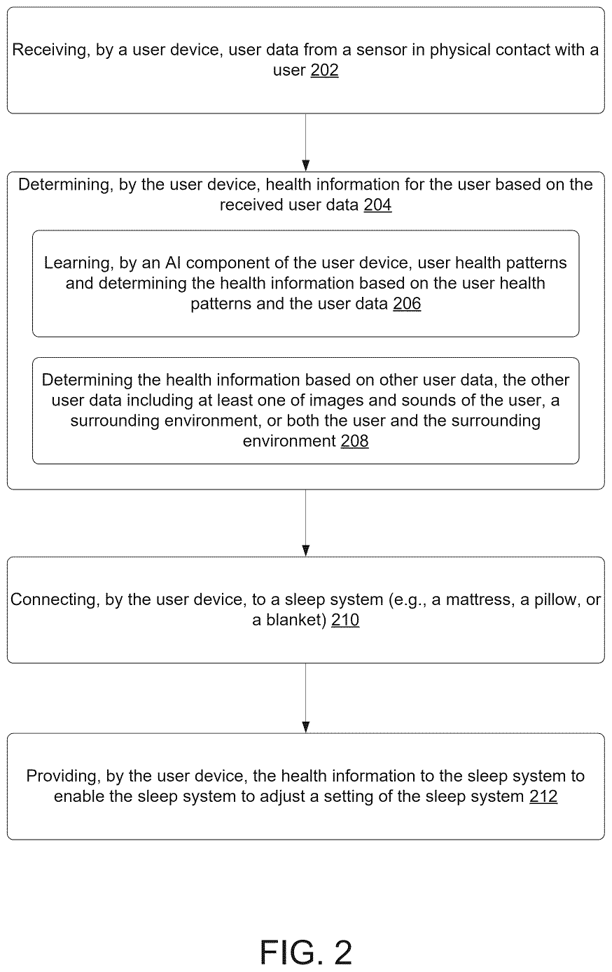

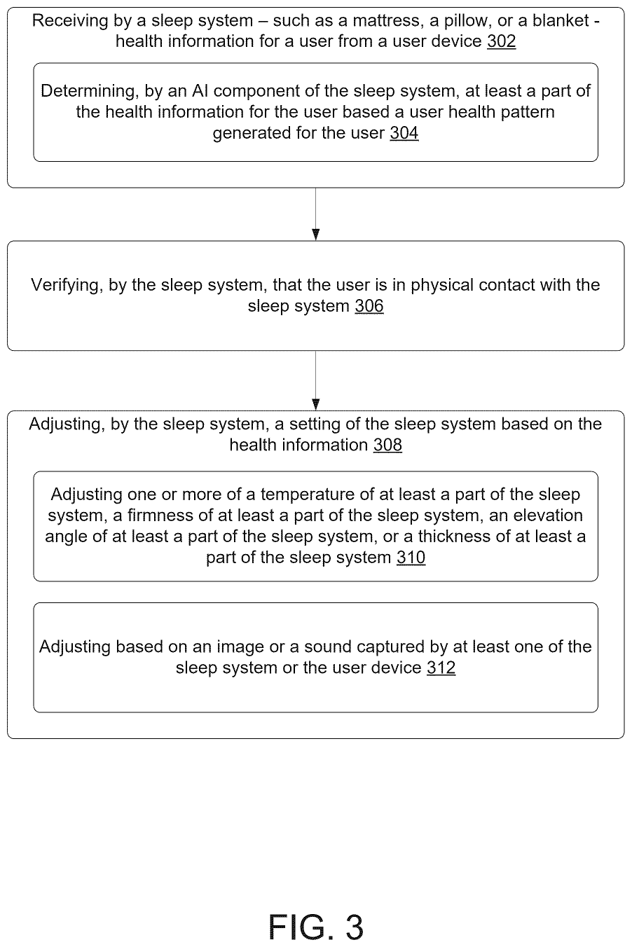

FIGS. 2 and 3 illustrate example processes. These processes are illustrated as logical flow graphs, each operation of which represents a sequence of operations that can be implemented in hardware, software, or a combination thereof. In the context of software, the operations represent computer-executable instructions stored on one or more computer-readable storage media that, when executed by one or more processors, perform the recited operations. Generally, computer-executable instructions include routines, programs, objects, components, data structures, and the like that perform particular functions or implement particular abstract data types. The order in which the operations are described is not intended to be construed as a limitation, and any number of the described operations can be omitted or combined in any order and/or in parallel to implement the processes.

FIG. 2 is a flow diagram of an illustrative process for a user device to determine health information for a user based on user data captured by a sensor physically contacting the user and to provide the health information to a sleep system, such as a mattress or pillow, to cause the sleep system to adjust a setting. As illustrated at 202, a user device may receive user data from a sensor in physical contact with a user. The user device may be an IoT device or a UE in communication with an IoT device and the sensor may be part of an IoT device. In some implementations, the user data may include at least one of a heartrate, a blood pressure, an electrocardiogram reading, an oxygen level a temperature, or a respiratory rate.

At 204, the user device may determine health information for the user based on the received user data. At 206, the user device may include or communicate with an AI component that learns user health patterns and determines the health information based on the user health patterns and the user data. In some implementations, the AI component may include an LLM trained with additional user data of the user or user data of a plurality of users. At 208, the user device may further determine the health information based on other user data, the other user data including at least one of images and sounds of the user, a surrounding environment, or both the user and the surrounding environment.

At 210, the user device may connect to a sleep system (e.g., at least one of a mattress or pillow). In some examples, the sleep system may be a guest mattress or pillow accepting connections and health information from multiple different user devices.

At 212, the user device may provide the health information to the sleep system to enable the sleep system to adjust a setting of the sleep system. The setting may include at least one of a firmness setting, an elevation angle setting of part of the sleep system (e.g., part of the mattress or pillow), an elevation angle setting of all of the sleep system, a thickness setting of the sleep system, or a temperature setting of the sleep system. In one example, the sleep system is a mattress, the user data indicates an elevated blood pressure, and the setting is an elevation angle of a bottom portion of the mattress.

FIG. 3 is a flow diagram of an illustrative process for a sleep system to receive health data for a user captured by a sensor physically contacting the user and to adjust a setting of the sleep system based on the health information. As illustrated at 302, a sleep system, such as at least one of a mattress or a pillow, may receive health information for a user from a user device. The health information may be determined by the user device based on user data captured by a sensor physically contacting the user. At 304, the sleep system may comprise an AI component that determines at least a part of the health information for the user based a user health pattern generated for the user.

At 306, the sleep system may verify that the user is in physical contact with the sleep system.

At 308, the sleep system may adjust a setting of the sleep system based on the health information. At 310, the adjusting may include adjusting one or more of a temperature of at least a part of the sleep system, a firmness of at least a part of the sleep system, an elevation angle of at least a part of the sleep system, or a thickness of at least a part of the sleep system. At 312, the adjusting may further be based on an image or a sound captured by at least one of the sleep system or the user device.

FIG. 4 is a schematic diagram of a computing device capable of implementing functionality of at least one of the user device or sleep system described herein. As shown, the computing device 400 includes a memory 402 storing modules and data 404, processor(s) 406, transceivers 408, and input/output devices 410.

In various examples, the memory 402 can include system memory, which may be volatile (such as RAM), non-volatile (such as ROM, flash memory, etc.) or some combination of the two. The memory 402 can further include non-transitory computer-readable media, such as volatile and nonvolatile, removable and non-removable media implemented in any method or technology for storage of information (e.g., having information stored thereon), such as computer readable instructions, data structures, program modules, or other data. System memory, removable storage, and non-removable storage are all examples of non-transitory computer-readable media. Examples of non-transitory computer-readable media include, but are not limited to, RAM, ROM, EEPROM, flash memory or other memory technology, CD-ROM, digital versatile discs (DVD) or other optical storage, magnetic cassettes, magnetic tape, magnetic disk storage or other magnetic storage devices, or any other non-transitory medium which can be used to store the desired information.

The memory 402 can include one or more software or firmware elements, such as computer-readable instructions that are executable by the one or more processors 406. For example, the memory 402 can store computer-executable instructions associated with modules and data 404. The modules and data 404 can include a platform, operating system, and applications, and data utilized by the platform, operating system, and applications. Further, the modules and data 404 can implement any of the functionality for the devices and components described and illustrated herein.

In various examples, the processor(s) 406 can be a central processing unit (CPU), a graphics processing unit (GPU), or both CPU and GPU, or any other type of processing unit. Each of the one or more processor(s) 406 may have numerous arithmetic logic units (ALUs) that perform arithmetic and logical operations, as well as one or more control units (CUs) that extract instructions and stored content from processor cache memory, and then executes these instructions by calling on the ALUs, as necessary, during program execution. The processor(s) 406 may also be responsible for executing all computer applications stored in the memory 402, which can be associated with types of volatile (RAM) and/or nonvolatile (ROM) memory.

The transceivers 408 can include modems, interfaces, antennas, Ethernet ports, cable interface components, and/or other components that perform or assist in exchanging wireless communications, wired communications, or both.

While the computing device need not include input/output devices 410, in some implementations it may include one, some, or all of these. For example, the input/output devices 410 can include a display, such as a liquid crystal display or any other type of display. For example, the display may be a touch-sensitive display screen and can thus also act as an input device or keypad, such as for providing a soft-key keyboard, navigation buttons, or any other type of input. The input/output devices 410 can include any sort of output devices known in the art, such as a display, speakers, a vibrating mechanism, and/or a tactile feedback mechanism. Output devices can also include ports for one or more peripheral devices, such as headphones, peripheral speakers, and/or a peripheral display. The input/output devices 410 can include any sort of input devices known in the art. For example, input devices can include a microphone, a keyboard/keypad, and/or a touch-sensitive display, such as the touch-sensitive display screen described above. A keyboard/keypad can be a push button numeric dialing pad, a multi-key keyboard, or one or more other types of keys or buttons, and can also include a joystick-like controller, designated navigation buttons, or any other type of input mechanism.

Although features and/or methodological acts are described above, it is to be understood that the appended claims are not necessarily limited to those features or acts. Rather, the features and acts described above are disclosed as example forms of implementing the claims.

Also, while the descriptions provided herein may be in the context of certain radio access technologies, networks, and network topologies, such as Fifth Generation (5G)/new radio (NR) mobile communications, the proposed concepts, schemes, and any variations thereof may be implemented in, for and by other types of radio access technologies, networks, and network topologies. Such radio access technologies, networks, and network topologies may include, for example and without limitation, Long-Term Evolution (LTE), Internet-of-Things (IOT), Narrow Band Internet of Things (NB-IOT), vehicle-to-everything (V2X), fixed wireless internet, and NTN communications. Thus, the scope of the disclosure is not limited to the examples described herein.

What is claimed is:

1. A method comprising:
determining, by a user device, health information for a user based on user data captured by a sensor physically contacting the user; and
providing, by the user device, the health information to at least one of a mattress or pillow to enable the at least one of the mattress or pillow to adjust a setting of the at least one of the mattress or pillow.

2. The method of claim 1, wherein the user device is an Internet-of-Things (IOT) device or a user equipment (UE) in communication with an IoT device and the sensor is part of an IoT device.

3. The method of claim 1, wherein the user data includes at least one of a heartrate, a blood pressure, an electrocardiogram reading, an oxygen level a temperature, or a respiratory rate.

4. The method of claim 1, wherein the setting includes at least one of a firmness setting, an elevation angle setting of part of the mattress or pillow, an elevation angle setting of all of the mattress or pillow, a thickness setting of the mattress or pillow, or a temperature setting of the mattress or pillow.

5. The method of claim 1, wherein the mattress or pillow is a mattress, the user data indicates an elevated blood pressure, and the setting is an elevation angle of a bottom portion of the mattress.

6. The method of claim 1, wherein the user device includes or communicates with an artificial intelligence (AI) component that learns user health patterns and determines the health information based on the user health patterns and the user data.

7. The method of claim 6, wherein the AI component includes a large language model (LLM) trained with additional user data of the user or user data of a plurality of users.

8. The method of claim 1, wherein the determining includes further determining the health information based on other user data, the other user data including at least one of images and sounds of the user, a surrounding environment, or both the user and the surrounding environment.

9. The method of claim 1, further comprising connecting, by the user device, to the at least one of the mattress or pillow, wherein the at least one of the mattress or pillow is a guest mattress or pillow accepting connections and health information from multiple different user devices.

10. A user device comprising:
a sensor for capturing user data when in physical contact with a user;
one or more processors; and
programming instructions that, when executed by the one or more processors, cause the user device to perform operations including:
determining health information for the user based on user data captured by the sensor when in physical contact with the user; and
providing the health information to a sleep system to enable the sleep system to adjust a setting of the sleep system.

11. The user device of claim 10, wherein the user data includes at least one of a heartrate, blood pressure, an electrocardiogram reading, an oxygen level, a temperature, or a respiratory rate.

12. The user device of claim 10, wherein the setting includes at least one of a firmness setting, an elevation angle setting of part of the sleep system, an elevation angle setting of all of the sleep system, a thickness setting of the sleep system, or a temperature setting of the sleep system.

13. The user device of claim 10, further comprising an artificial intelligence (AI) component included in the user device or in communication with the user device, wherein the AI component is configured to learn user health patterns and determine the health information based on the user health patterns and the user data.

14. The user device of claim 10, wherein the operations further include connecting to the sleep system, wherein the sleep system is a guest mattress or pillow accepting connections and health information from multiple different user devices.

15. A non-transitory computer storage medium having stored thereon programming instructions that, when executed by one or more processors of a sleep system, cause the sleep system to perform operations comprising:
receiving health information for a user from a user device, wherein the health information is determined based on user data captured by a sensor physically contacting the user; and
adjusting a setting of the sleep system based on the health information.

16. The non-transitory computer storage medium of claim 15, wherein the sleep system includes at least one of a mattress a pillow, or a blanket.

17. The non-transitory computer storage medium of claim 15, wherein the operations further comprise verifying that the user is in physical contact with the sleep system before performing the adjusting.

18. The non-transitory computer storage medium of claim 15, wherein the adjusting comprises adjusting one or more of a temperature of at least a part of the sleep system, a firmness of at least a part of the sleep system, an elevation angle of at least a part of the sleep system, or a thickness of at least a part of the sleep system.

19. The non-transitory computer storage medium of claim 15, wherein the adjusting is further based on an image or a sound captured by at least one of the sleep system or the user device.

20. The non-transitory computer storage medium of claim 15, wherein the sleep system comprises an artificial intelligence (AI) component that is stored on the non-transitory computer storage medium and that, when operated by the one or more processors, determines health information for the user based a user health pattern generated for the user.
